(19) United States
(12) Patent Application Publication
(10) Pub. No.: US 2011/0015274 A1
(43) Pub. Date: Jan. 20, 2011

(54) FORM OF AN AMINOINDAN MESYLATE DERIVATIVE

(76) Inventors: Ester Masllorens Llinas, Girona (ES); Ernesto Duran Lopez, Barcelona (ES)

Correspondence Address: IP Patent Docketing;K&L GATES LLP, 599 Lexington Avenue, 33rd Floor, New York, NY, 10022-6030, US

(21) Appl. No.: 12/839,980

(22) Filed: Jul. 20, 2010

Related U.S. Application Data
(60) Provisional application No. 61/226,793, filed on Jul. 20, 2009.

(51) Int. Cl. A61K 31/135; C07C 211/33; A61P 25/16

(52) U.S. Cl. 514/657; 564/428

(57) ABSTRACT

The present disclosure relates generally to rasagiline mesylate with improved flowability and with advantageously reduced stickiness, to processes for the preparation thereof, and to the use thereof for milling and for preparing pharmaceutical formulations. Methods for improving the flowability and/or alleviating the stickiness of rasagiline mesylate having a very poor flowability and/or showing undesirable stickiness are also disclosed.

### CROSS REFERENCE TO RELATED ED APPLICATION

This application claims priority to U.S. Provisional Application No. 61/226,793, filed 20 Jul. 2009, which application is expressly incorporated herein by reference in its entirety.

### TECHNICAL FIELD

The present disclosure relates generally to rasagiline mesylate with improved flowability and with advantageously reduced stickiness, to processes for the preparation thereof, and to the use thereof for milling and for preparing pharmaceutical formulations. Methods for improving the flowability and/or alleviating the stickiness of rasagiline mesylate having a very poor flowability and/or showing undesirable stickiness are also disclosed.

### RELEVANT BACKGROUND

Rasagiline mesylate is an active pharmaceutical substance with an empirical formula of C₁₂H₁₃N.CH₄O₃S and a molecular weight of 267.34. Rasagiline mesylate is the international commonly accepted name for R-(+)-N-propargyl-1-aminoindan mesylate (or (1R)-N-prop-2-yn-1-ylindan-1-amine-mesylate or (1R)-2,3-dihydro-N-2-propynyl-1H-inden-1-amine mesylate), which is represented in Formula I.

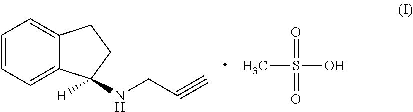

Rasagiline mesylate is an active substance indicated for the treatment of the signs and symptoms of idiopathic Parkinson's disease as initial monotherapy and as adjunct therapy to levodopa. Rasagiline is a selective irreversible inhibitor of the B-form of monoamine oxidase enzyme (MAO-B). In the United States, rasagiline mesylate is marketed under the name Azilect™ for the treatment of early Parkinson's disease.

The preparation of rasagiline mesylate is described in U.S. Pat. No. 5,532,415. More precisely, in Example 6B of this reference the product is obtained by treating the enantiopure rasagiline L-tartrate salt with methanesulfonic acid in isopropanol at reflux temperature for 30 minutes, allowing the reaction to cool to room temperature, and filtering the resulting precipitate. Further, in Example 31 it is described that the said rasagiline mesylate showed excellent chemical stability and kept an appearance of white powder when submitted to accelerated thermal degradation conditions (i.e. heating at 80° C. for 72, 96 or 144 hours, and refluxing in isopropanol for 30 hours). However, U.S. Pat. No. 5,532,415 does not provide any information regarding the stability of said rasagiline mesylate after extended storage under humid (stressing) conditions (e.g., 40° C., 75% relative humidity for up to four weeks).

Rasagiline mesylate is known to be a moderately hygroscopic material. For example, International Patent Application Publication No. WO 2008/019871 describes that rasagiline mesylate in powder form transforms into a sticky powder when stored under humid conditions (i.e., 40° C., 75% relative humidity). In particular, Example 5 of WO 2008/019871 describes that although no substantial changes in the water content of the samples were detected, rasagiline mesylate agglomerates after 4 weeks at 60° C. in a closed glass bottle and transforms into a sticky powder after 4 weeks at 40° C., 75% relative humidity in an opened container. Additionally, Example 8 of WO 2008/019871 describes that rasagiline mesylate was prepared by reaction of rasagiline base with methanesulfonic acid in isopropanol and has the following particle size distribution: d(0.1)=13.6±0.05 μm, d(0.5)=40.5±0.25 μm, d(0.9)=85.7±0.89 μm, with 100% of particles below 250 μm. WO 2008/019871 does not disclose the specific surface area of the obtained rasagiline mesylate.

International Patent Application Publication No. WO 2007/061717 further describes an alternative conversion of rasagiline L-tartrate to rasagiline mesylate. In particular, Example 17 of this reference describes the preparation of rasagiline mesylate by isolating rasagiline base from rasagiline tartrate, followed by treating the obtained rasagiline base with methanesulfonic acid in isopropanol to obtain a suspension of rasagiline mesylate, heating the suspension at reflux temperature to dissolve the solid, cooling to 10° C. to crystallize rasagiline mesylate, and filtering the precipitate.

Rasagiline mesylate as thus obtained by the processes disclosed in the above discussed prior art, which all make use of isopropanol as a crystallization solvent, shows very poor flowability characteristics and also shows stickiness after storage under humid (stressing) conditions. Precisely, rasagiline mesylate crystals obtained by crystallization processes using isopropanol as a solvent have a Hausner ratio (hereinafter referred to as “HR”) greater than or equal to about 1.67, which indicates an extremely poor flow character (see also Reference Examples 1 and 2 below). Also, the rasagiline mesylate crystals obtained by crystallization processes using isopropanol as a solvent show a particle size distribution having a d(0.9) of less than about 250 μm and a specific surface area higher than about 0.20 m²/g (again see also Reference Examples 1 and 2 below).

Flowability affects the ease with which a material is handled during processing into a pharmaceutical product. Namely, when flowability is very poor, problems may occur with handling and processing during the milling and formulating. Compressibility index (or Carr Index, hereinafter referred to as “CI”) and the closely related HR are well accepted methods for measuring the powder flow of a pharmaceutical powder (See European Pharmacopoeia 6.6, 2.9.36 Powder flow). The CI is a measure of the propensity of a powder to consolidate, and so it is a measure of the relative importance of inter-particulate interactions, which is especially relevant in poor flowing materials. Although the CI method cannot be used as a sole measure of powder flowability, it has the advantage of being simple to calculate, and it provides a quick comparison between API, excipients, and formulations (See Developing Solid Oral Dosage Forms Pharmaceutical Theory & Practice, Academic Press 2009. Y. Qiu, L. Liu, Y. Chen, G. G. Z. Zhang, W. Porter). The basic procedure to measure the CI and the HR is to measure the unsettled apparent volume, (V₀), and the final tapped volume, (V_{f}), of the powder after tapping the material until no further volume changes occur. The CI and the HR are calculated as follows:

Compressibility Index (or Carr Index)=100×[(V₀−V_{f})/V₀]

Hausner ratio=V₀/V_{f}

Alternatively, the CI and the HR can be calculated using measured values of bulk density (ρ_{bulk}) and tapped density (ρ_{tapped}), as follows:

Compressibility Index (or Carr Index)=100×[(ρ_{tapped}−ρ_{bulk})/ρ_{tapped}]

Hausner ratio=ρ_{tapped}/ρ_{bulk}

For the CI and the HR, the generally accepted scale of flowability is given in Table 2.9.36.-2 of European Pharmacopoeia as follows:

| Compressibility | | |
|---|---|---|
| index (per cent) | Flow Character | Hausner Ratio |
| 1-10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| >38 | Very, very poor | >1.60 |

A low HR and a low CI indicate a high flowability. In this regard, it is generally accepted that a HR greater than or equal to 1.46 and a CI greater than or equal to 32 indicate a very poor flowing material which is rarely acceptable for manufacturing purposes. Therefore, a HR less than 1.46 and a CI less than 32 indicate an acceptable flowing material. In this regard, since the unit dose amount of rasagiline mesylate is quite low relative to the total weight of the tablet (typically, an amount of rasagiline mesylate equivalent to 0.5 or 1 mg of rasagiline base is present in a tablet with total weight of over 200 mg), then a decrease in the flowability of the rasagiline mesylate could result in a large percent deviation from the required amount to be present in a tablet.

Also, a rasagiline mesylate having a CI value below 32 as compared with a CI value greater than or equal to 32 indicates that the former rasagiline mesylate shows improved compressibility characteristics, which means that it shows better propensity to consolidate and is therefore more suitable for tablet formation.

International Patent Application Publication No. WO 2006/091657 describes the provision of certain particle size distributions of rasagiline and in particular discloses a mixture of particles of a pharmaceutically acceptable salt of rasagiline, preferably rasagiline mesylate, having a d(0.9) or D₉₀ of less than 250 μm. This d(0.9)/D₉₀ for rasagiline should provide rasagiline with a particle size distribution similar to that disclosed by hereinbefore discussed International Patent Application Publication No. WO 2008/019871 having 100% of particles below 250 μm. As discussed in WO 2008/019871, rasagiline with a majority of particles having a particle size below 250 μm can result in agglomeration and transformation into a sticky powder under the storage conditions disclosed. Further, although not explicitly disclosed, rasagiline mesylate crystals having a d(0.9) or D₉₀ of less than 250 μm are expected to have a specific surface area higher than about 0.20 m²/g.

Additionally, according to International Patent Application Publication No. WO 2006/091657, it is desirable to provide such particle size distributions for rasagiline pharmaceutically acceptable salts with a view to obtaining an improved content uniformity of resulting pharmaceutical compositions, due to the relatively low unit dose amount of rasagiline mesylate relative to the total weight of the tablet. WO 2006/091657 also teaches that particles of rasagiline as obtained from salt crystallization are large and irregular, which can easily decrease content uniformity. WO 2006/091657 thus teaches that mechanical comminution is the specific process which provides rasagiline having a d(0.9) or D₉₀ of less than 250 microns, which leads to an improved content uniformity of the tablet. In particular, Example 1 of WO 2006/091657 discloses the milling of rasagiline mesylate samples containing large, irregular particles (i.e. allegedly, obtained from crystallization), which initially showed a d(0.9) or D₉₀ of between 386-598 μm, and, after milling, showed a d(0.9) or D₉₀ of between 156-189 μm (i.e. d(0.9) or D₉₀ below 250 μm).

International Patent Application Publication No. WO 2009/122301 is a co-pending application that describes provision of rasagiline mesylate having a D₉₀ in the range of 600 to 1500 μm, which is obtained by dissolving rasagiline mesylate in a solvent medium comprising an ester solvent and an alcoholic solvent, subjecting the solution to gradual cooling, and optionally seeding the cooled solution. WO 2009/122301 also describes rasagiline mesylate having a D₉₀ in the range of 255 to 1400 μm as obtained from milling the former rasagiline mesylate having a D₉₀ in the range of 600 to 1500 μm, and also pharmaceutical compositions having a D₉₀ in the range of 255 to 1500 μm. But WO 2009/122301 does not discuss or specifically address the issues of agglomeration and/or stickiness known to be associated with prior art rasagiline mesylate as referred to above.

Rasagiline mesylate crystals having a D₉₀ in the range of 600 to 1500 μm obtained by the process described in International Patent Application Publication No. WO 2009/122301 are big crystals which are not suitable for pharmaceutical use due to the low unit dose amount of rasagiline mesylate, and indeed the crystals are described to be useful as starting material for preparing milled rasagiline mesylate. Furthermore, rasagiline mesylate crystals having a D₉₀ in the range of 600 to 1500 μm obtained by the process described in WO 2009/122301 show a specific surface area of less than 0.03 m²/g. This specific surface area is illustrated in Reference Example 3 below, which shows a rasagiline mesylate having a D₉₀ of 611 μm and a specific surface area of 0.0274 m²/g, as obtained following the process of Example 1 WO 2009/122301.

Rasagiline mesylate having a D₉₀ in the range of 255 to 1400 μm as obtained from milling in International Patent Application Publication No. WO 2009/122301 is expected to have a high specific surface area (i.e. greater than 0.20 m²/g). But the said rasagiline mesylate crystals cannot be unequivocally reproduced to measure the specific surface area since the milling process described therein is not enabling as it does not provide the specific milling conditions to be used. Namely, the said crystals are obtained by mechanical comminution (grinding), which usually creates considerable crystal defects and surface irregularities. Therefore, for a similar particle size and crystal shape, a higher specific surface area would be expected for crystals obtained by grinding when compared to crystals obtained by slow growing during crystallization.

International Patent Application Publication No. WO 2010/059913 is a co-pending application that describes provision of rasagiline mesylate having a D₉₀ of less than 6 μm. WO 2010/059913 also describes that rasagiline mesylate having a D₉₀ of less than 6 μm shows agglomeration properties when stored under humid conditions (i.e., 25±2° C., 65±5% relative humidity) during 5 days (see Example 33 of WO 2010/059913). Specifically for Example 33, it can be seen that the rasagiline mesylate on storage under humid conditions resulted in a D₉₀ of approximately 151 μm, indicating agglomeration.

Thus, in view of the foregoing there is a need to provide rasagiline mesylate with improved flowability and which also alleviates the problems associated with known rasagiline compositions on storage.

### BRIEF DESCRIPTION OF THE FIGURE

FIG. 1 illustrates the X-ray powder diffraction (XRD) of rasagiline mesylate Form I according to the present disclosure.

### SUMMARY OF THE DISCLOSURE

The disclosure relates generally to rasagiline mesylate with improved flowability and with advantageously reduced stickiness, to processes for the preparation thereof, and to the use thereof for milling and for preparing pharmaceutical formulations. Methods for improving the flowability and/or alleviating the stickiness of rasagiline mesylate having a very poor flowability and/or showing undesirable stickiness are also disclosed.

### DETAILED DESCRIPTION OF THE PREFERRED EMBODIMENTS

Reference will now be made in detail to certain preferred embodiments. This invention may, however, be embodied in many different forms and should not be construed as limited to the specific embodiments as set forth herein.

It will be apparent to those skilled in the art that various modifications and variations can be made to the present invention and specific examples provided herein without departing from the spirit or scope of the invention. Thus, it is intended that the present invention covers the modifications and variations of this invention that come within the scope of any claims and their equivalents.

In a first aspect, rasagiline mesylate, preferably crystals of rasagiline mesylate in the known crystalline Form I, with improved flowability characteristics (i.e. having a HR less than 1.46) and with improved compressibility characteristics (i.e. having a CI less than about 32) and which are therefore acceptable for manufacturing purposes, are disclosed.

The disclosed crystals of rasagiline mesylate have an improved flowability character as compared with the rasagiline mesylate crystals obtained in the prior art, which have a non-desirable extremely poor flowability (i.e. having a HR greater than or equal to about 1.67, and having a CI greater than or equal to about 40). Thus, the crystals of rasagiline mesylate with improved flowability described herein are better handled and processed during the milling and formulating of a product, as compared with the rasagiline mesylate crystals of the prior art. Consequently, the crystals of rasagiline mesylate described herein are more suitable for pharmaceutical formulation use.

Also, the disclosed crystals of rasagiline mesylate have a CI value below 32 as compared with the rasagiline mesylate crystals obtained in the prior art, which show a CI value greater than or equal to 40. Thus, the crystals of rasagiline mesylate disclosed herein show improved compressibility characteristics, which means that the crystals show better propensity to consolidate, and are therefore more suitable for tablet formation, as compared with the rasagiline mesylate crystals obtained in the prior art.

Measurement of HR is well-known in the art and is described, for example, by Mersmann; Crystallization Technology Handbook (A. Mersmann, ed., 2^{nd} ed., Marcel Dekker). In the present disclosure, the bulk and tapped densities for each sample of rasagiline mesylate were determined using a PT-TD1 tapped densitometer from Pharma Test. The HR of the rasagiline mesylate sample was calculated by dividing the tapped bulk density by the bulk density.

Measurement of CI is well-known in the art and is described, for example, by Y. Qiu, L. Liu, Y. Chen, G. G. Z. Zhang, W. Porter in Developing Solid Oral Dosage Forms Pharmaceutical Theory & Practice, Academic Press 2009. In the present disclosure, the bulk and tapped densities for each sample of rasagiline mesylate were determined using a PT-TD1 tapped densitometer from Pharma Test. The CI of the rasagiline mesylate sample was calculated by the following formula:

Compressibility Index (or Carr Index)=100×[(ρ_{tapped}−ρ_{bulk})/ρ_{tapped}]

The inventors have also surprisingly found that the rasagiline mesylate obtained by the processes disclosed herein and in greater detail below shows a considerably reduced specific surface area than the rasagiline mesylate obtained by the prior art processes which employ crystallization from isopropanol. The inventors have thus found that the specific surface area of rasagiline mesylate as prepared according to the present disclosure is especially affected by the specific crystallization process employed.

The present disclosure thus further provides rasagiline mesylate with advantageous specific surface area with a view to providing a product with improved flowability and compressibility, and in particular reduced stickiness and/or agglomeration. More specifically, there is now further provided rasagiline mesylate with a specific surface area in the range of about 0.03 m²/g to about 0.20 m²/g, preferably in the range of about 0.04 m²/g to about 0.18 m²/g and even more preferably in the range of about 0.05 m²/g to about 0.15 m²/g.

The inventors have still further surprisingly found that rasagiline mesylate in accordance with the present disclosure is completely stable under accelerated stability humid conditions, that is at 40° C. and 75% relative humidity, showing substantially no sticking and/or agglomeration when stored for up to 6 months under these conditions. Therefore, rasagiline mesylate as provided herein alleviates the above discussed sticking problems associated with the rasagiline mesylate of the prior art, which showed sticking and/or agglomeration when stored for 1 month under these conditions. There is, therefore, still further provided by the present disclosure rasagiline mesylate characterized by substantially no agglomeration when stored for at least 1 month at 40° C. and 75% relative humidity. The substantial absence of agglomeration in accordance with the present invention can be determined by visual inspection and/or by optical microscopy. Also, the substantial absence of agglomeration can be determined by particle size analysis that does not comprise sonication of the sample, wherein said particle size analysis confirms that the D₉₀ and/or the D₅₀ of the rasagiline mesylate has an increase of less than 100% of value after storage. That is, the smallest agglomerate to be observed by particle size analysis would be the agglomerate resultant of the union of a single crystal with another which would lead to an increase of 100% in the D₉₀ and/or the D₅₀ measured value.

Further, the rasagiline mesylate in accordance with the present disclosure does not show hygroscopic properties when stored at a temperature of 25° C. (±1° C.) and a relative humidity of 80% (±2%) during 24 hours.

It is generally understood in the pharmaceutical field that some materials exhibit a marked tendency to self adhere, or to adhere to a contact surface, which property is generally known as stickiness. The interaction of water with solid powders can be a prime cause of stickiness and caking in these solids. Therefore, stickiness of a solid material can be directly related to its wettability, which reflects whether water will spread on the particle surface as a continuous film or, conversely, retract as one or several drops. Consequently, a solid material having a good wettability will have a strong mutual affinity with an adherend surface, and thus is likely to adhere well (see, for example, Int. J. Food Prop. 2001, 4, 1-33). Similarly, a solid material having a good wettability will stick easier than a solid material having a reduced wettability.

Quantitative measurement of wettability is generally carried out using force tensiometry and optical tensiometry (also known as goniometry). These methods, however, have significant limitations when applied to powders and porous solids. The Washburn method for measuring contact angles by force tensiometry, for example, depends on a material constant which reflects the porosity of the solid and the packing of particles. Because this value is assumed to be constant, any actual variation generates error in the calculation of contact angles and therefore in the quantitative measurement of powder wettability.

But a quantitative measurement of wettability is not necessary for analysis of the stickiness and/or agglomeration properties of a powder solid. The effective surface area (i.e. the area of the particle which becomes wetted) is known to be proportional to the specific surface area of the particle and the specific surface area can be easily measured by gas adsorption techniques, using the BET (Brunauer, Emmett and Teller) isotherm. This method also has the advantage of measuring the surface of fine structures and deep texture on the particles.

It is also well known in the art that particle size distribution is not directly related either to the specific surface area of the particles, or to the effective surface area, since any assumption about the particle shape fails to account for surface texture of the particles. For example, see Reference Example 2 and Example 1, in which rasagiline mesylate crystals having a similar particle size distribution, i.e. a D₁₀ of about 14-31 μm, a D₅₀ of about 60-89 μm, and a D₉₀ of about 143-164 μm, show very different specific surface areas. For example, the surface properties can be strongly affected by high energy input associated to mechanical comminution processes for the reduction of particle size. In this way, the high energy input can cause a disruption of the crystal lattice on the particle surface and thus create crystal defects, which can result in an increased specific surface area when compared to particles having a similar particle size distribution but obtained by a controlled crystallization process. Furthermore, the well-ordered surface of the crystallized material becomes disordered during the mechanical comminution process and therefore amorphous regions can be formed. Such amorphous structures can show an increased affinity to water vapour compared with crystalline surfaces (see Pharm. Dev. Technol. 2004, 9, 1-13), thus increasing the wettability and consequently the stickiness of the powder material. Accordingly, stickiness and/or agglomeration properties cannot be meaningfully predicted based on particle size distribution.

Rasagiline mesylate as provided by the present disclosure can be further characterised by particle size measurements, and in a particularly preferred embodiment rasagiline mesylate is characterised by a D₉₀ in the range of about 100 to 600 μm.

It is surprising that rasagiline mesylate with a particle size as above is particularly suitable for pharmaceutical formulation into a low-dose dosage form as employed in accordance with the present disclosure based on the approved dosage regimen for rasagiline. More specifically, rasagiline mesylate is approved as 0.5 and 1 mg (base equivalent amount) tablets for the treatment of idiopathic Parkinson's disease.

It is generally accepted practice in the formulation field that for such low-dose dosage forms that it is particularly important to formulate APIs with a controlled particle size distribution so as to ensure content uniformity and homogeneity. In particular, there are recognized calculations known in the art to drug formulators that ensure that particle size, in particular mean particle diameter, provides a 99% probability of a resulting formulation passing the USP content uniformity test.

Specifically for rasagiline mesylate based on such recognized particle size calculations known in the art, it can be shown that it would be expected and routinely calculated that rasagiline mesylate should have a D₉₀ value well below 250 μm to exhibit the required content uniformity as required by the USP content uniformity test. There thus existed, based on the approved dosage form of rasagiline, a general prejudice against formulating rasagiline mesylate with a D₉₀ as now employed in accordance with the present disclosure. It is thus even more surprising that rasagiline mesylate is now obtained with a D₉₀ as described herein and that this rasagiline mesylate according to the present disclosure exhibits advantageous flow and compressibility properties that facilitate the provision of rasagiline mesylate formulations with required content uniformity. Further, a product which will show advantageously reduced stickiness properties during formulation will exhibit a better content uniformity.

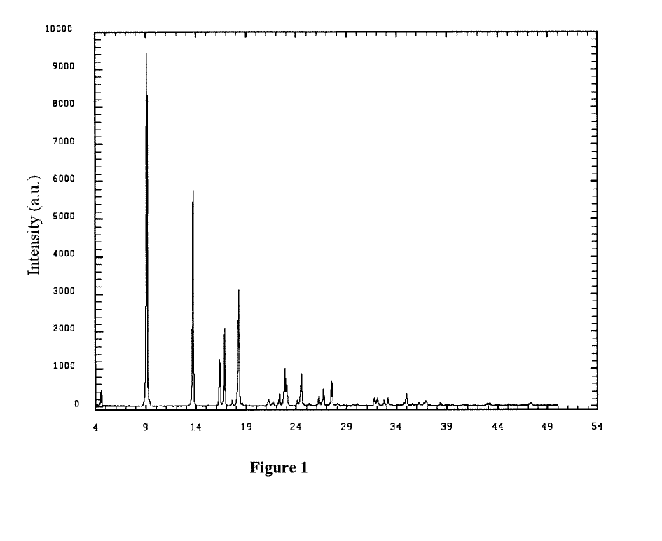

Another aspect of the present disclosure relates to a process for preparing crystals of rasagiline mesylate with improved flowability and improved compressibility as associated with the present disclosure, and in this respect it is further understood that rasagiline mesylate as described herein preferably comprises rasagiline mesylate in crystalline form (known Form I), wherein said crystalline form shows an X-ray powder diffraction pattern comprising peaks at about 4.7, 9.0, 13.5, 14.2, 15.1, 16.2, 16.6, 17.4, 18.1, 21.1, 21.5, 22.1, 22.7, 22.9, 23.9, 24.3, 25.1, 26.1, 26.5, 27.3 and 33.0°±0.2 degrees 2θ (See FIG. 1).

According to the present disclosure, therefore, there is further provided a process comprising (i) providing a hot solution of rasagiline mesylate in a solvent comprising acetonitrile; (ii) cooling the hot solution to a temperature in the range of about 70 to about 72° C. at a controlled mean cooling rate less than or equal to approximately 3° C./min; (iii) optionally, stirring at this temperature for at least 30 minutes; (iv) cooling to a temperature in the range of about 20 to about 25° C. at a controlled mean cooling rate less than or equal to approximately 3° C./min to obtain a suspension; (v) optionally, stirring the suspension at this temperature for a suitable time; (vi) optionally, cooling the suspension to a temperature in the range of about 0 to about 5° C.; (vii) optionally, stirring the suspension at this temperature for a suitable time; and (viii) isolating the crystals of rasagiline mesylate having a HR less than 1.46 and a CI less than 32 from the suspension substantially as hereinbefore described, and/or having a specific surface area in the range of about 0.03 m²/g to about 0.20 m²/g substantially as hereinbefore described, and/or exhibiting substantial lack of agglomeration substantially as hereinbefore described.

Surprisingly, the inventors have found that by carrying out the process above, the crystals of rasagiline mesylate obtained exhibit an unexpected characteristic, i.e. show an improved flowability and improved compressibility and furthermore show substantially no stickiness properties, as compared with the rasagiline mesylate crystals obtained in the prior art.

Acetonitrile is the preferred solvent for step (i) of the process described herein. Step (i) is preferably carried out at reflux to provide the hot solution of rasagiline mesylate.

The cooling rate value is calculated as the variation of Celsius degrees temperature per minute, and it is expressed as the absolute value.

The applicants have found that by controlling the cooling of the hot solution of rasagiline mesylate in a solvent comprising acetonitrile as described above, the crystals of rasagiline mesylate obtained exhibit a higher improved flowability and improved compressibility, as compared with the rasagiline mesylate crystals obtained in the prior art (i.e. show a HR less than about 1.35 and a CI less than about 32).

Accordingly, there is further provided by the present disclosure rasagiline mesylate obtained, or obtainable, by a process substantially as herein described. Such rasagiline mesylate exhibits advantageous properties again substantially as herein described.

In another aspect, the use of rasagiline mesylate with Unproved flowability and improved compressibility and with advantageously reduced stickiness properties as a starting material for preparing a composition comprising rasagiline mesylate with reduced particle size by means of mechanical size reduction procedures (i.e. comminution), preferably by means of milling procedures, is disclosed. Since the rasagiline mesylate disclosed herein shows an improved flowability nature and an advantageously reduced stickiness as compared with the rasagiline mesylate obtained in the prior art, handling and processing during the milling of a composition comprising the same to reduce the particle size is also easier and more efficient. For example, rasagiline mesylate according to the present disclosure does not stick to milling and/or formulation equipment, which is clearly desirable during the formulation process.

In yet another aspect, the use of rasagiline mesylate with improved flowability and improved compressibility and with advantageously reduced stickiness properties is disclosed for preparing a pharmaceutical formulation comprising rasagiline mesylate. Since the disclosed rasagiline mesylate shows an improved flowability and compressibility nature and an advantageously reduced stickiness as compared with the rasagiline mesylate obtained in the prior art, handling and processing during the formulation of the pharmaceutical composition comprising the same is also easier and more efficient, especially for tablet formation.

In another further aspect, the present disclosure relates to a method for improving the flowability of rasagiline mesylate crystals with non-acceptable very poor flowing character (i.e. having a HR greater than or equal to 1.46) comprising crystallizing the rasagiline mesylate crystals in a solvent comprising acetonitrile as described above. Also, the present disclosure relates to a method for improving the compressibility characteristics of rasagiline mesylate crystals with a CI greater than or equal to 32 comprising crystallizing the rasagiline mesylate crystals in a solvent comprising acetonitrile as described in the process above. Additionally, the present disclosure relates to a method for reducing the stickiness of rasagiline mesylate, which method comprises crystallizing the rasagiline mesylate crystals in a solvent comprising acetonitrile as described above, so as to obtain rasagiline mesylate characterized by substantially no agglomeration when stored for at least 1 month at 40° C. and 75% relative humidity, wherein said absence of agglomeration has been determined by visual inspection, by optical microscopy, and/or by particle size analysis that does not comprise sonication of the sample, wherein said particle size analysis confirms that the D₉₀ of the rasagiline mesylate has an increase of less than 100% of value after the storage.

There is also provided by the present disclosure a pharmaceutical composition comprising an effective amount of rasagiline mesylate substantially as hereinbefore described, together with a pharmaceutically acceptable carrier, diluent or excipient therefor. The term “effective amount” as used herein means an amount of rasagiline mesylate which is capable of preventing, ameliorating or eliminating the signs and symptoms of idiopathic Parkinson's disease. By “pharmaceutically acceptable carrier, diluent or excipient” is meant that the carrier, diluent or excipient must be compatible with rasagiline mesylate and not be deleterious to a recipient thereof. Suitable pharmaceutically acceptable compositions according to the present invention are preferably tablets.

The present disclosure further provides rasagiline mesylate substantially as hereinbefore described for use in the treatment of Parkinson's disease or for the manufacture of a medicament for the treatment of Parkinson's disease. The present disclosure also provides a method of treatment of Parkinson's disease, which method comprises administering to the patient an effective amount of rasagiline mesylate substantially as hereinbefore described.

The particle size parameters measured in the present disclosure have been obtained by means of laser light diffraction technique, and specifically by means of a Malvern Mastersizer S particle size analyzer having characteristics as set out below. Namely, the laser source used was a 2 milliwatt Helium/neon laser (633 nm wavelength); the detection system was a Fourier Transform lens system; the sample was run using a 2.40 mm lens; the sample unit was a sample unit for wet measurement, and particularly was a MS1-Small volume Sample Dispersion Unit stirred cell. The wet dispersion was prepared by using a solution of 1.5 g of Soybean Lecithin in 200 mL of Isopar G as a sample dispersant, and the dispersion was controlled by stirring the unit cell. Regarding the analyzed samples, these were prepared by wetting a weighed amount of rasagiline mesylate (approximately 200 mg) with few drops of sample dispersant, introducing the sample with a spatula to the previously background and corrected measuring cell filled with dispersant (Isopar G) until the obscuration reached the desired level, and sonicating the sample for 3 minutes at 5 watts. The characterization parameters (volume distributions) were measured for at least six times for each sample, and the result is a mean of said measured values for each sample.

The specific surface area values disclosed in the present disclosure have been obtained by means of a specific surface area analysis technique based on the BET (Brunauer, Emmett and Teller) theory, which is a well-accepted theory known in the art for the calculation of surface areas of solids by means of measuring their physical adsorption of gas molecules (See S. Brunauer, P. H. Emmett and E. Teller, J. Am. Chem. Soc., 1938, 60, 309). In particular, the specific surface area values measured have been calculated from the BET surface area plot obtained by measuring the quantity of nitrogen gas molecules adsorbed by a weighted amount of solid at different relative pressures (P/P₀) within the range 0.05 to 0.3, at 77K. Precisely, the measurement of the adsorption of gas molecules was carried out by means of a Micromeritics™ Gemini V equipment having the characteristics as set out below. Namely, the gas used for adsorption measure was nitrogen gas. The analyzed sample was prepared by weighting rasagiline mesylate (approximately 1.2±0.3 g). The sample for analysis was degassed (at 30° C. for 10 minutes and at 130° C. for 90 minutes). The determination of the adsorption of nitrogen was measured at 77K and for twelve relative pressures sufficiently dispersed within in the range of 0.05 to 0.3 (i.e. twelve absolute pressures in the range of 38.860001 mmHg to 230.464996 mmHg relative to a saturated pressure of 765.705017 mmHg).

The term “rasagiline mesylate crystals which substantially do not show agglomeration” is understood to define crystals of rasagiline mesylate which do not substantially show agglomerates as determined by visual inspection and/or by optical microscopy. Also, it is understood to define crystals of rasagiline mesylate which do not substantially show agglomeration as determined by particle size analysis which does not comprise sonication of the sample wherein said particle size analysis confirms that the D₉₀ and/or the D₅₀ of the rasagiline mesylate crystals has an increase of less than 100% of value.

The following Examples are for illustrative purposes only and are not intended, nor should they be interpreted, to limit the scope of the claims.

### EXAMPLES

### General Experimental Conditions

### Particle Size Distribution Method:

The particle size for rasagiline mesylate was measured using a Malvern Mastersizer S particle size analyzer with an MS1-Small Volume Sample Dispersion Unit stirred cell. A 300RF mm lens and a beam length of 2.4 mm were used. Preparation of sample dispersant: 1.5 g of Soybean Lecithin was added to 200 mL of Isopar G, and the mixture was mixed gently until Lecithin dissolved. Samples for analysis were prepared by wetting a weighed amount of rasagiline mesylate (approximately 200 mg) with a few drops of sample dispersant to obtain a paste. The paste was delivered to the previously background and corrected measuring cell filled with dispersant (Isopar G) until the obscuration reached the desired level. The suspension was sonicated for 3 minutes. Volume distributions were obtained for at least six measures. After completing the measurements, the sample cell was emptied and cleaned, refilled with suspending medium, and the sampling procedure repeated again. For characterization, the values of D₁₀, D₅₀ and D₉₀ (by volume) were specifically listed, each one being the mean of the measured values available for each characterization parameter.

The notation Dₓ [also written as D(v, 0.X)] means that X % of the particles have a diameter less than a specified diameter D. Thus a D₉₀ [or D(v, 0.9)] of 100 μm means that 90% of the particles have a diameter less than 100 μm.

### Specific Surface Area Method:

The BET (Brunauer, Emmett and Teller) specific surface area for rasagiline mesylate was measured using a Micromeritics™ GEMINI V equipment (GEMINI CONFIRM V2.00 Software™). The sample for analysis was degassed at 30° C. for 10 minutes and at 130° C. for 90 minutes. The determination of the adsorption of N₂ at 77 K was measured for relative pressures in the range of 0.05 to 0.3 for a weighed amount of rasagiline mesylate (i.e., approximately 1.2±0.3 g).

### X-ray Powder Diffraction (XRD):

The XRD diffractogram was obtained using a RX SIEMENS D5000 diffractometer with a vertical goniometer, a copper anodic tube, and radiation CuK_{α}, λ=1, 54056 Á.

HPLC method:

The chromatographic separation was carried out in a Chiralpak IC, 5 μm, 250×4.6 mm I.D column; at 30° C.

The mobile phase was prepared by mixing 950 mL of n-hexane, 40 mL of 2-propanol, 10 mL of ethanol, 4 mL of trifluoroacetic acid and 1 mL of diethylamine. The mixture was mixed thoroughly.

The chromatograph was equipped with a 265 nm detector, and the flow rate was 1.4 mL per minute.

The test samples were prepared by dissolving the appropriate amount of sample to obtain 10 mg per mL in diluent. The diluent was prepared by mixing 89 mL of mobile phase, 10 mL of 2-propanol and 1 mL of diethylamine. The injection volume was 5 μL.

### Specific Examples

The rasagiline mesylate used in the following examples was prepared by following the processes described in Examples 1 to 3 of International Patent Application Publication No. WO 2009/141737.

### Reference Example 1

### Preparation of Rasagiline Mesylate Crystals

9.04 g of rasagiline mesylate and 36 mL of isopropanol were heated to reflux, until complete dissolution occurred, and stirred for 30 minutes at reflux. After this time, the mixture was cooled down to 0-5° C. and stirred for 30 minutes. The suspension was then filtered, and the collected solid was washed with 10 mL of isopropanol and dried at 50° C. for 4 h under vacuum. 8.66 g of white solid were thus obtained (95.80% yield).

Analytical data: Particle size: D(v, 0.1): 17.4 μm, D(v, 0.5): 62.6 μm, D(v, 0.9): 118.0 μm.; Bulk density (g/mL): 0.236; Tapped density (g/mL): 0.407; HR: 1.72; CI: 42; Specific Surface Area (BET): 0.3216 m²/g.

### Reference Example 2

### Preparation of Rasagiline Mesylate Crystals

10.13 g of rasagiline mesylate and 40.5 mL of isopropanol were heated to reflux until complete dissolution occurred. The solution was then allowed to cool down to 0-5° C. with the following cooling profile: cool to 70-72° C. at 1° C./min and stirred for 30 min at this temperature; cool to 20-25° C. at 1° C./min and stirred for 45 min at this temperature; cool to 0-5° C. at 1° C./min and stirred for at least 1 h at this temperature. The suspension was then filtered, and the collected solid was washed with 10 mL of isopropanol and dried at 50° C. for 4 h under vacuum. 9.53 g of white solid were thus obtained (94.08% yield).

Analytical data: Particle size: D(v, 0.1): 14.7 μm, D(v, 0.5): 60.2 μm, D(v, 0.9): 143.6 μm; Bulk density (g/mL): 0.259; Tapped density (g/mL): 0.432; HR: 1.67; CI: 40; Specific Surface Area (BET): 0.2490 m²/g.

### Reference Example 3

### Preparation of Rasagiline Mesylate Crystals

Rasagiline mesylate (5 g) was added to ethyl acetate (150 mL), and the mixture was heated to reflux, followed by the addition of methanol (20 mL), to form a clear solution, and then stirred for 15 minutes. The resulting solution was slowly cooled to 60° C. with slow stirring. The stirring was stopped and the reaction mass was maintained for 1 hour at same temperature to grow the crystals. The resulting mass was further cooled to 45-50° C. and kept for 12 hours at 45-50° C. The resulting mass was finally cooled to 25-30° C., the material was filtered and then dried under vacuum at 60° C. to produce 4.9 g of rasagiline mesylate.

Analytical data: Particle size: D(v, 0.1): 127.5 μm, D(v, 0.5): 311.2 μm, D(v, 0.9): 611.1 μm.; Specific Surface Area: 0.0274 m²/g.

### Example 1

### Preparation of Rasagiline Mesylate Crystals with Improved Flowability and Compressibility (i.e. HR<1.46 and CI<32)

130.35 g of rasagiline mesylate and 519 mL of acetonitrile were heated to reflux until complete dissolution occurred and stirred for 30 minutes at reflux. The solution was then allowed to cool down to 20-25° C. and stirred for 1 h at this temperature. After this time, the suspension was cooled down to 0-10° C. and stirred for 2 h. The suspension was then filtered, and the collected solid was washed with 2×50 mL of acetonitrile and dried at 50° C. under vacuum until constant weight. 113.76 g of white solid were thus obtained (87.27% yield).

Analytical data: Particle size: D(v, 0.1): 30.6 μm, D(v, 0.5): 89.0 μm, D(v, 0.9): 163.6 μm; Bulk density (g/mL): 0.386; Tapped density (g/mL): 0.551; HR: 1.43; CI: 30; Specific Surface Area (BET): 0.0722 m²/g.

### Example 2

### Preparation of Rasagiline Mesylate Crystals with Improved Flowability and Compressibility (i.e. HR<1.46 and CI<32)

112.38 g of rasagiline mesylate and 454 mL of acetonitrile were heated to reflux until complete dissolution occurred and stirred for 30 minutes at reflux. The solution was then allowed to cool down to 20-25° C. and stirred for 1 h at this temperature. After this time, the suspension was cooled down to 0-10° C. and stirred for 2 h. The suspension was then filtered, and the collected solid was washed with 2×40 mL of acetonitrile and dried at 50° C. under vacuum until constant weight. 107.03 g of white solid were thus obtained (95.24% yield).

Analytical data: Particle size: D(v, 0.1): 37.0 μm, D(v, 0.5): 118.7 μm, D(v, 0.9): 246.7 μm; Bulk density (g/mL): 0.416; Tapped density (g/mL): 0.586; HR: 1.41; CI: 29.

### Example 3

### Preparation of Rasagiline Mesylate Crystals with Improved Flowability and Compressibility (i.e. HR<1.46 and CI<32)

65.76 g of rasagiline mesylate and 265 mL of acetonitrile were heated to reflux until complete dissolution occurred. The solution was then allowed to cool down to 0-5° C. with the following cooling profile: cool to 70-72° C. at 1° C./min and stirred for 30 min at this temperature; cool to 20-25° C. at 1° C./min and stirred for 45 min at this temperature; cool to 0-5° C. at 1° C./min and stirred for at least 1 h at this temperature. The suspension was then filtered, and the collected solid was washed with 2×60 mL of acetonitrile and dried at 50° C. for 4 h under vacuum. 61.79 g of white solid were thus obtained (93.96% yield).

Analytical data: Particle size: D(v, 0.1): 37.4 μm, D(v, 0.5): 159.6 μm, D(v, 0.9): 404.2 μm; Bulk density (g/mL): 0.256; Tapped density (g/mL): 0.346; HR: 1.35; CI: 26; Specific Surface Area (BET): 0.0474 m²/g.

### Example 4

### Preparation of Rasagiline Mesylate Crystals with Improved Flowability and Compressibility (i.e. HR<1.46 and CI<32)

0.497 Kg of rasagiline mesylate and 1.73 Kg of acetonitrile were heated to reflux until complete dissolution occurred. The solution was then cooled to 68-74° C. in not less than 15 min and stirred for at least 30 min at this temperature; cooled to 20-25° C. in not less than 1 h and stirred for at least 45 min at this temperature; cooled to 0-5° C. in not less than 15 min and stirred for at least 1 h at this temperature. The suspension was then filtered, and the collected solid was washed with 2×0.38 Kg of acetonitrile and dried at 50° C.±2° C. for 8 h under vacuum. 0.431 Kg of white solid were thus obtained (86.72% yield).

Analytical data: Particle size: D(v, 0.1): 54.9 μm, D(v, 0.5): 208.5 μm, D(v, 0.9): 464.0 μm; Bulk density (g/mL): 0.497; Tapped density (g/mL): 0.710; HR: 1.43; CI: 30; Specific Surface Area (BET): 0.1434 m²/g; Powder X-Ray Diffraction: Form I (see FIG. 1).

### Example 5

### Stability Studies of Rasagiline Mesylate

Rasagiline mesylate obtained in Example 4 was exposed to air moisture under controlled conditions (40±2° C., 75%±5% relative humidity) for 6 months. Samples were taken and analyzed after 1, 2, 3 and 6 months storage.

| | Initial | | | | |
|---|---|---|---|---|---|
| | sample | 1 month | 2 months | 3 months | 6 months |
| Description | White | White | White | White | White |
| | to off | to off | to off | to off | to off |
| | white | white | white | white | white |
| | powder. | powder. | powder. | powder. | powder. |
| Agglomer- | Not | Not | Not | Not | Not |
| ation (visual | observed | observed | observed | observed | observed |
| observation) | | | | | |
| Total | <0.05% | <0.05% | <0.05% | <0.05% | <0.05% |
| impurities | | | | | |
| (HPLC) | | | | | |

### Example 6

### Hygroscopicity Test

Rasagiline Mesylate Obtained in Example 4 was Stored at 25° C.±1° C. and 80%±2% relative humidity during 24 hours. The percentage increase in mass was calculated based on the hygroscopicity method described in the European Pharmacopoeia 5.0 (general chapter 5.11). Result: % Mass increase: 0.01 (i.e. not hygroscopic material).

Although the invention has been described and illustrated with a certain degree of particularity, it is understood that the disclosure has been made only by way of example and that numerous changes in the conditions and order of steps can be resorted to by those skilled in the art without departing from the spirit and scope of the invention.

The invention is, of course, not limited to the examples described but cover all the variants defined in the claims. The terms “a” and “an” and “the” and similar referents used in the context of the following claims are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g. “such as”) provided herein is intended merely to better illuminate the embodiments and does not pose a limitation on the scope of the embodiments otherwise claimed. No language in the specification should be construed as indicating any non-claimed element essential to the practice of the embodiments.

The use of the term “or” in the claims is used to mean “and/or” unless explicitly indicated to refer to alternatives only or the alternatives are mutually exclusive, although the disclosure supports a definition that refers to only alternatives and “and/or.” Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventors expect those of ordinary skill in the art to employ such variations as appropriate, and the inventors intend for the embodiments to be practiced otherwise than specifically described herein. Accordingly, these embodiments include all modifications and equivalents of the subject matter recited in the claims appended hereto as permitted by applicable law. Moreover, any combination of the above-described elements in all possible variations thereof are encompassed by the embodiments unless otherwise indicated herein or otherwise clearly contradicted by context.

Further, it is to be understood that the example embodiments disclosed herein are illustrative. Other modifications that may be employed are within the scope of the embodiments. Thus, by way of example, but not of limitation, alternative configurations of the present embodiments may be utilized in accordance with the teachings herein. Accordingly, the present embodiments are not limited to that precisely as shown and described in the specification and figure.

1. Rasagiline mesylate 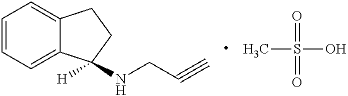
having a Hausner ratio less than about 1.46 and a Carr Index less than about 32.

2. The rasagiline mesylate of claim 1, wherein said rasagiline mesylate has a Hausner ratio of about 1.12 to about 1.45 and a Carr Index of about 11 to about 31.

3. The rasagiline mesylate of claim 2, wherein said rasagiline mesylate has a Hausner ratio of about 1.19 to about 1.45 and a Carr Index of about 16 to about 31.

4. The rasagiline mesylate of claim 3, wherein said rasagiline mesylate has a Hausner ratio of about 1.26 to about 1.45 and a Carr Index of about 21 to about 31.

5. The rasagiline mesylate of claim 4, wherein said rasagiline mesylate has a Hausner ratio of about 1.35 to about 1.45 and a Carr Index of about 26 to about 31.

6. Rasagiline mesylate having a specific surface area of about 0.03 m²/g to about 0.20 m²/g.

7. The rasagiline mesylate of claim 6, wherein said rasagiline mesylate has a specific surface area of about 0.04 m²/g to about 0.18 m²/g.

8. The rasagiline mesylate of claim 7, wherein said rasagiline mesylate has a specific surface area of about 0.05 m²/g to about 0.15 m²/g.

9. The rasagiline mesylate of claim 1 having a specific surface area of about 0.03 m²/g to about 0.20 m²/g.

10. The rasagiline mesylate of claim 5 having a specific surface area of about 0.05 m²/g to about 0.15 m²/g.

11. Rasagiline mesylate characterized by substantially no agglomeration when stored for at least 1 month at 40° C. and 75% relative humidity.

12. The rasagiline mesylate of claim 11, wherein said absence of agglomeration has been determined by at least one of visual inspection, optical microscopy, and particle size analysis of a sample that does not comprise sonication of the sample, and wherein particle size analysis confirms that the D₉₀ and/or the D₅₀ of the rasagiline mesylate has an increase of less than 100% of value after the storage.

13. The rasagiline mesylate of claim 1, wherein said rasagiline mesylate has a D₉₀ of about 100 to 600 μm.

14. The rasagiline mesylate of claim 6, wherein said rasagiline mesylate has a D₉₀ of about 100 to 600 μm.

15. The rasagiline mesylate of claim 1, wherein said rasagiline mesylate is in crystalline form and wherein said crystalline form is characterized by an X-ray powder diffraction pattern comprising peaks at about 4.7, 9.0, 13.5, 14.2, 15.1, 16.2, 16.6, 17.4, 18.1, 21.1, 21.5, 22.1, 22.7, 22.9, 23.9, 24.3, 25.1, 26.1, 26.5, 27.3 and 33.0°±0.2 degrees 2θ.

16. The rasagiline mesylate of claim 6, wherein said rasagiline mesylate is in crystalline form and wherein said crystalline form is characterized by an X-ray powder diffraction pattern comprising peaks at about 4.7, 9.0, 13.5, 14.2, 15.1, 16.2, 16.6, 17.4, 18.1, 21.1, 21.5, 22.1, 22.7, 22.9, 23.9, 24.3, 25.1, 26.1, 26.5, 27.3 and 33.0°±0.2 degrees 2θ.

17. A process for preparing crystals of the rasagiline mesylate of claim 1, the process comprising:
(i) providing a hot solution of rasagiline mesylate in a solvent comprising acetonitrile,
(ii) cooling the hot solution to a temperature of about 70 to about 72° C. at a controlled mean cooling rate equal to or less than approximately 3° C./min,
(iii) further cooling the solution to a temperature of about 20 to about 25° C. at a controlled mean cooling rate equal to or less than approximately 3° C./min to obtain a suspension,
(vi) isolating crystals of rasagiline mesylate from the suspension.

18. The process of claim 17 further comprising at least one of:
(a) stirring the solution of step (ii) at that temperature for at least 30 minutes,
(b) stirring the suspension of step (iii) at that temperature for a suitable time,
(c) further cooling the suspension of step (iii) to a temperature of about 0 to about 5° C., and
(d) stirring the cooled suspension of step (iii)(c) at that temperature for a suitable time.

19. The process of claim 17, wherein the solvent comprising acetonitrile of step (i) is acetonitrile.

20. The process of claim 17, wherein step (i) comprises refluxing rasagiline mesylate in the solvent comprising acetonitrile.

21. A process for preparing crystals of the rasagiline mesylate of claim 6, the process comprising:
(i) providing a hot solution of rasagiline mesylate in a solvent comprising acetonitrile,
(ii) cooling the hot solution to a temperature of about 70 to about 72° C. at a controlled mean cooling rate equal to or less than approximately 3° C./min,
(iii) further cooling the solution to a temperature of about 20 to about 25° C. at a controlled mean cooling rate equal to or less than approximately 3° C./min to obtain a suspension,
(vi) isolating crystals of rasagiline mesylate from the suspension.

22. The process of claim 21 further comprising at least one of:
(a) stirring the solution of step (ii) at that temperature for at least 30 minutes,
(b) stirring the suspension of step (iii) at that temperature for a suitable time,
(c) further cooling the suspension of step (iii) to a temperature of about 0 to about 5° C., and
(d) stirring the cooled suspension of step (iii)(c) at that temperature for a suitable time.

23. The process of claim 21, wherein the solvent comprising acetonitrile of step (i) is acetonitrile.

24. The process of claim 21, wherein step (i) comprises refluxing rasagiline mesylate the a solvent comprising acetonitrile.

25. A pharmaceutical composition comprising an effective amount of the rasagiline mesylate of claim 1, together with a pharmaceutically acceptable carrier, diluent or excipient therefor.

26. A pharmaceutical composition comprising an effective amount of the rasagiline mesylate of claim 6, together with a pharmaceutically acceptable carrier, diluent or excipient therefor.

27. A method of treatment of Parkinson's disease, the method comprising administering to a patient an effective amount of the rasagiline mesylate of claim 1.

28. A method of treatment of Parkinson's disease, the method comprising administering to a patient an effective amount of the rasagiline mesylate of claim 6.
